(19) United States
(12) Patent Application Publication
(10) Pub. No.: US 2026/0048019 A1
(43) Pub. Date: Feb. 19, 2026

(54) USE OF CO-PROCESSED EXCIPIENTS IN CONTINUOUS MANUFACTURING OF SOLID DOSAGE FORMS

(71) Applicant: Hoffmann-La Roche Inc., Little Falls, NJ (US)

(72) Inventors: Davide DE FRANCO, Liestal (CH); Andreas EWERT, Buggingen (DE); Emmanuela GAVI, Basel (CH); Reto MAURER, Basel (CH)

(73) Assignee: Hoffmann-La Roche Inc., Little Falls, NJ (US)

(21) Appl. No.: 19/103,173

(22) PCT Filed: Aug. 10, 2023
(86) PCT No.: PCT/EP2023/072135

§ 371 Date: Feb. 11, 2025

(30) Foreign Application Priority Data
Aug. 12, 2022 (EP) 22190194.5
Jan. 31, 2023 (EP) 23154126.9

(51) Int. Cl. A61K 9/20; A61K 9/28; A61K 31/4985; A61K 31/501; A61K 31/5377

(52) U.S. Cl. CPC A61K 9/2095; A61K 9/2018; A61K 9/2054; A61K 9/2059; A61K 9/282; A61K 9/2893; A61K 31/4985; A61K 31/501; A61K 31/5377

(57) ABSTRACT

The present invention relates to the use of co-processed excipients in continuous manufacturing of solid dosage forms.

### FIELD OF THE INVENTION

The present invention relates to the use of co-processed excipients in continuous manufacturing of solid dosage forms.

### BACKGROUND OF THE INVENTION

Continuous pharmaceutical manufacturing offers potential flexibility, quality, and economic advantages over batch processing (Sau L. Lee et al., J. Pharm. Innov. 2015, 10, 191-199). However, the number of feeders on devices that are used in continuous manufacturing are limited, typically to four to six feeders. Consequently, continuous manufacturing of solid pharmaceutical dosage forms, such as tablets, is limited to compositions consisting of an API and only three to five excipients, unless pre-blends of multiple excipients are used. The use of pre-blends is, however, economically inefficient, thus partly defeating the advantages of continuous manufacturing. Therefore, there is a need to simplify pharmaceutical compositions in a way that they consist of as few excipients as possible.

### SUMMARY OF THE INVENTION

The inventors of the present invention have found that an effective way to simplify compositions is to introduce co-processed excipients, which combine the functionalities of multiple single excipients. Thus, in one aspect, the present invention relates to the use of co-processed excipients in continuous manufacturing of solid dosage forms.

### BRIEF DESCRIPTION OF THE FIGURES

FIG. 1 depicts a flow chart of the continuous mini-batch direct compression process according to the invention described in Example 1.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

As used herein, the term “co-processed excipient” relates to any combination of 2 or more excipients obtained by physical co-processing that does not lead to the formation of covalent bonds. Co-processed excipients have functionalities that are not achievable through sample blending. Co-processed excipients can be produced by processes that produce only a physical interaction between the components, like, for example, co-drying, spray drying, granulation, extrusion, and high-shear dispersion. Examples of co-processed excipients include, but are not limited to, Di-Pac®, Emdex®, Pharmatose®, Sugar Tab®, Pharmaburst 500®, TIMERx®, Ludipress®, Starlac®, Xylitab®, StarCap®, Advantose®, Ludiflash®, Cellactose®, ForMaxx®, Microcelac 100®, Avicel®, ProSolv® SMCC, ProSolv Easytab®, Combilac®, Startab®, Parteck® ODT, Comprecel SMCC 90, Pharmacel SMCC 90, SANAQ ML 011, and SANAQ SP205.

The term “Di-Pac®” refers to a co-processed excipient consisting of co-crystallized sucrose (97%) and maltodextrin (3%).

The term “Emdex®” refers to a co-processed excipient consisting of 95% glucose monohydrate and 5% oligosaccharides resulting from the enzymatic hydrolysis of starch.

The term “Pharmatose®” refers to an excipient consisting of crystalline lactose monohydrate.

The term “Sugar Tab®” refers to a co-processed excipient consisting of sucrose (90% to 93%) and invert sugar (7% to 10%).

The term “Pharmaburst 500®” refers to a co-processed excipient consisting of mannitol (75% to 90%), sorbitol (6% to 20%), crospovidone (7% to 15%) and silicon dioxide (0.1% to 1.5%).

The term “TIMERx®” refers to a co-processed excipient consisting of xanthan gum, locust bean gum, and dextrose.

The term “Ludipress®” refers to a co-processed excipient consisting of 93% lactose monohydrate, 3.5% povidone having a K-value of 30 (“Kollidon® 30”) and 3.5% crospovidone having a bulk density of 0.30-0.40 g/mL (“Kollidon® CL”).

The term “Starlac®” refers to a co-processed excipient that is made from lactose and maize starch.

The term “Xylitab®” refers to an excipient consisting of xylitol.

The term “StarCap®” refers to a co-processed excipient that is made from pregelatinized starch and maize starch.

The term “Advantose®” refers to a co-processed excipient consisting of spray-dried fructose and starch.

The term “Ludiflash®” refers to a co-processed excipient consisting of 84.0-92.0% D-mannitol, 4.0-6.0% Kollidon® CL-SF, 3.5-6.0% polyvinyl acetate, 0.5-2.0% water and 0.25-0.60% povidone.

The term “Kollidon® CL-SF” refers to an excipient consisting of crospovidone.

The term “Cellactose®” refers to a co-processed excipient obtained by spray drying of 75% α-lactose monohydrate and 25% of cellulose powder.

The term “ForMaxx®” refers to a co-processed excipient consisting of calcium carbonate and sorbitol.

The term “Microcelac 100®” refers to a co-processed excipient obtained by spray drying 75% α-lactose monohydrate and 25% microcrystalline cellulose.

The term “Avicel®” refers to a co-processed excipient obtained by spray drying microcrystalline cellulose and carboxymethylcellulose sodium.

The term “SMCC90” refers to a co-processed excipient obtained by spray drying 98% microcrystalline cellulose and 2% colloidal silicon dioxide.

The term “ProSolv® Easytab” refers to a co-processed excipient consisting of microcrystalline cellulose (96%), sodium starch glycolate (1.2%), colloidal silicon dioxide (2%), and sodium stearyl fumarate (0.8%).

The term “Combilac®” refers to a co-processed excipient consisting of 70% alpha-lactose monohydrate, 20% microcrystalline cellulose (MCC) and 10% white, native corn starch.

The term “Startab®” refers to an excipient consisting of starch.

The term “Parteck® ODT” refers to a co-processed excipient consisting of D-mannitol and croscarmellose sodium.

The term “SANAQ ML 011” refers to a co-processed excipient consisting of lactose monohydrate and microcrystalline cellulose.

The term “SANAQ SP205” refers to a co-processed excipient consisting of microcrystalline cellulose, colloidal silicon dioxide, crospovidone, and povidone.

As used herein, the term “API” refers to an active pharmaceutical ingredient. Preferably, the API is a small molecule, i.e. an organic compound having a molecular weight of ≤1000 daltons. Particular, yet non-limiting examples of APIs are ralmitaront, alogabat, and fenebrutinib.

As used herein, the term “filler” refers to a substance added to a pharmaceutical composition to increase the weight and/or size of the pharmaceutical composition. Pharmaceutically acceptable fillers are described in Remington's Pharmaceutical Sciences and listed in Handbook of Pharmaceutical Excipients, Sheskey et al., 2017. Non-limiting examples of fillers are starch (e.g., pregelatinized starch), cellulose (e.g., microcrystalline cellulose) and lactose (e.g., lactose monohydrate). Preferred, yet non-limiting examples of fillers are cellulose and lactose.

As used herein, the term “disintegrant” refers to a substance added to a pharmaceutical composition to help break apart (disintegrate), e.g., after administration, and release the active ingredient, such as Form B described herein. Pharmaceutically acceptable disintegrants are described in Remington's Pharmaceutical Sciences and listed in Handbook of Pharmaceutical Excipients, Sheskey et al., 2017. Non-limiting examples of disintegrants are low substituted hydroxypropyl cellulose (also known as hydroxypropyl methylcellulose (HPMC) or hypromellose) and croscarmellose sodium. A preferred, yet non-limiting example of a disintegrant is croscarmellose sodium.

As used herein, the term “acidulant” refers to a pharmaceutically acceptable excipient having a pH of 1% (w/w) aqueous solution of less than 4.0. The acidulant is usually added to enhance the taste or to improve the dissolution of (basic) APIs. Some examples of acidulants include citric acid, tartaric acid, fumaric acid, lactic acid, malic acid, succinic acid, phosphoric acid and acetic acid. Preferably, the acidulant is selected from the group consisting of citric acid, tartaric acid, fumaric acid, lactic acid and/or malic acid. More preferably, the acidulant is fumaric acid.

As used herein, the term “lubricant” refers to a substance added to a pharmaceutical composition to help reduce the adherence of a granule of powder to equipment surfaces. Pharmaceutically acceptable glidants are described in Remington's Pharmaceutical Sciences and listed in Handbook of Pharmaceutical Excipients, Sheskey et al., 2017. Non-limiting examples of glidants are sodium stearyl fumarate and magnesium stearate. A preferred, yet non-limiting example of a glidant is sodium stearyl fumarate.

As used herein, the term “flow agent” refers to a substance added to a pharmaceutical composition to enhance product flow by reducing interparticulate friction. Pharmaceutically acceptable flow agents are described in Remington's Pharmaceutical Sciences and listed in Handbook of Pharmaceutical Excipients, Sheskey et al., 2017. Non-limiting examples of flow agents include silicon dioxide (colloidal), polyethylene glycol PEG 6000, fumed silicon dioxide Aerosil® 200, talc and the like. A preferred, yet non-limiting example is silica, colloidal anhydrous.

As used herein, the term “crospovidone” refers to crosslinked homopolymer of N-vinyl-2-pyrrolidinone.

Particular, yet non-limiting examples of “sugar alcohols” as used herein include mannitol and isomalt.

As used herein, the term “mini-batch” refers to a variation of a batch blending process whereby the size of each batch is reduced to minimize the mass of material ‘in-process’. Series of discrete Mini-Batches are transferred onto a conventional rotary tablet press enabling a continuous tablet manufacturing via direct compression process.

As used herein, the term “direct compression” refers to a tablet manufacturing process, where physically mixed powder blends of pharmaceutical active ingredient(s) (API) and excipients are directly compressed to tablets without the addition of a wet or dry granulation step.

As used herein, the term “flowability” refers to the ability of a bulk powder to flow in a piece of equipment. It is quantified with appropriate testing devices such as the shear tester. Usually the ratio ff_{c} of consolidation stress, to unconfined yield strength, is used to characterize flowability numerically.

As used herein, the term “bulk density” refers to the ratio of the mass of an amount of bulk solid to its volume. It is typically measured by gently introducing a known sample mass into a graduated cylinder, and carefully leveling the powder without compacting it. The apparent untapped volume is then read to the nearest graduated unit.

As used herein, the term “ralmitaront” refers to 5-ethyl-4-methyl-N-[4-[(2S) morpholin-2-yl]phenyl]-1H-pyrazole-3-carboxamide.

As used herein, the term “alogabat” refers to 6-[[5-methyl-3-(6-methyl-3-pyridyl) isoxazol-4-yl]methoxy]-N-tetrahydropyran-4-yl-pyridazine-3-carboxamide.

As used herein, the term “fenebrutinib” refers to 2-[3′-(Hydroxymethyl)-1-methyl-5-([5-[(2S)-2-methyl-4-(oxetan-3-yl)piperazin-1-yl]pyridin-2-yl]amino)-6-oxo-1,6-dihydro-3,4′-bipyridin-2′-yl]-7,7-dimethyl-3,4,7,8-tetrahydro-2H-cyclopenta[4,5]pyrrolo[1,2-a]pyrazin-1 (6H)-one.

### New Uses of Co-Processed Excipients

In a first aspect, the present invention provides the use of co-processed excipients in continuous manufacturing of solid dosage forms.

In a preferred embodiment, said continuous manufacturing is continuous mini-batch direct compression.

In one embodiment, said solid dosage form is a tablet comprising:
(i) a kernel; and optionally
(ii) a coating.

As outlined above, the number of feeders on devices that are used in continuous manufacturing are limited, typically to four to six feeders. Thus, it is important to limit the number of excipients in pharmaceutical compositions if they are to be manufactured in a continuous fashion.

In one embodiment, said kernel consists of:
(i) an API;
(ii) 1-4 a co-processed excipients; and
(iii) 1-4 further excipients selected from fillers, disintegrants, lubricants, flow agents, and acidulants;
wherein the total number of excipients (ii) and (iii) is ≤5.

In one embodiment, said kernel consists of:
(i) an API;
(ii) a co-processed excipient;
(iii) a filler, disintegrant or an acidulant; and
(iv) a lubricant.

In one embodiment, said filler is selected from starch, cellulose, sugar alcohols, calcium phosphate and lactose.

In one embodiment, said disintegrant is selected from low substituted hydroxypropyl cellulose, crospovidone, sodium starch glycolate and croscarmellose sodium.

In one embodiment, said acidulant is fumaric acid.

In one embodiment, said lubricant is selected from sodium stearyl fumarate, polyethylene glycol and magnesium stearate.

In one embodiment, said flow agents are selected from colloidal silicon dioxide, polyethylene glycol PEG 6000, fumed silicon dioxide Aerosil® 200, and talc.

In one embodiment, said co-processed excipient is selected from Di-Pac®, Emdex®, Pharmatose®, Sugar Tab®, Pharmaburst 500®, TIMERx®, Ludipress®, Starlac®, Xylitab®, StarCap®, Advantose®, Ludiflash®, Cellactose®, ForMaxx®, Microcelac 100®, Avicel®, ProSolv® SMCC90, Prosolv Easytab®, Combilac®, Startab®, Parteck® ODT, Comprecel SMCC 90, Pharmacel SMCC 90, SANAQ ML 011, and SANAQ SP205.

In a preferred embodiment, said co-processed excipient is selected from Ludipress®, Microcelac, ProSolv SMCC90®, ProSolv Easytab®, Combilac®, and Startab®.

In a preferred embodiment, said co-processed excipient is selected from Combilac and ProSolv SMCC90.

In a particularly preferred embodiment, said co-processed excipient is Ludipress®.

In a particularly preferred embodiment, said co-processed excipient is Microcelac®.

In a particularly preferred embodiment, said co-processed excipient is ProSolv SMCC90®.

In a particularly preferred embodiment, said co-processed excipient is ProSolv Easytab®.

In a particularly preferred embodiment, said co-processed excipient is Combilac®.

In a particularly preferred embodiment, said co-processed excipient is Startab®.

In a preferred embodiment, said API is selected from ralmitaront, alogabat, and fenebrutinib.

In a particularly preferred embodiment, said API is ralmitaront.

In a particularly preferred embodiment, said API is alogabat.

In a particularly preferred embodiment, said API is fenebrutinib.

### New Tablet Blends

In a further aspect, the present invention provides a blend for continuous direct compression of tablet kernels, consisting of:
(i) an API;
(ii) a co-processed excipient; and
(iii) 1-4 further excipients selected from fillers, disintegrants, lubricants, and flow agents.

In one embodiment, said blend consists of:
(i) an API;
(ii) a co-processed excipient;
(iii) a filler or a disintegrant; and
(iv) a lubricant.

In one embodiment, said filler is selected from starch, cellulose, sugar alcohols, calcium phosphate and lactose.

In one embodiment, said disintegrant is selected from low substituted hydroxypropyl cellulose, crospovidone, sodium starch glycolate and croscarmellose sodium.

In one embodiment, said acidulant is fumaric acid.

In one embodiment, said lubricant is selected from sodium stearyl fumarate, polyethylene glycol and magnesium stearate.

In one embodiment, said flow agents are selected from colloidal silicon dioxide, polyethylene glycol PEG 6000, fumed silicon dioxide Aerosil® 200, and talc.

In one embodiment, said co-processed excipient is selected from Di-Pac®, Emdex®, Pharmatose®, Sugar Tab®, Pharmaburst 500®, TIMERx®, Ludipress®, Starlac®, Xylitab®, StarCap®, Advantose®, Ludiflash®, Cellactose®, ForMaxx®, Microcelac 100®, Avicel®, ProSolv® SMCC90, Prosolv Easytab, Combilac®, Startab®, Parteck® ODT, Comprecel SMCC 90, Pharmacel SMCC 90, SANAQ ML 011, and SANAQ SP205.

In a preferred embodiment, said co-processed excipient is selected from Ludipress®, Microcelac®, ProSolv SMCC90®, ProSolv Easytab®, Combilac®, and Startab®.

In a preferred embodiment, said co-processed excipient is selected from Combilac and ProSolv SMCC90.

In a particularly preferred embodiment, said co-processed excipient is Ludipress®.

In a particularly preferred embodiment, said co-processed excipient is Microcelac®.

In a particularly preferred embodiment, said co-processed excipient is ProSolv SMCC90®.

In a particularly preferred embodiment, said co-processed excipient is ProSolv Easytab®.

In a particularly preferred embodiment, said co-processed excipient is Combilac®.

In a particularly preferred embodiment, said co-processed excipient is Startab®.

In a preferred embodiment, said API is selected from ralmitaront, alogabat, and fenebrutinib.

In a particularly preferred embodiment, said API is ralmitaront.

In a particularly preferred embodiment, said API is alogabat.

In a particularly preferred embodiment, said API is fenebrutinib.

It was found that, in order to facilitate free flowing material which can be gravimetrically fed onto the tablet press and to ensure a robust tablet compression process, it is important that the tablet blend has a flowability of >FFc 4-5. Thus, in a preferred embodiment, the blend according to the invention has a flowability of >FFc 4-5.

Furthermore, it was found that a blend having a bulk density of >0.4 g/mL improves the reliability of the tablet compression process. Thus, in a preferred embodiment, the blend according to the invention has a bulk density of >0.4 g/mL.

It was found that blends according to the invention can reliably be pressed into tablets in a continuous fashion even with high drug loads. In one embodiment, the blend according to the invention has a drug load of 1-30% wt/wt, preferably of 2-25% wt/wt, more preferably of 2-20% wt/wt.

In a particularly preferred embodiment, API (i) is ralmitaront; co-processed excipient (ii) is ProSolv SMCC 90; further excipient (iii) is a disintegrant being croscarmellose sodium; and lubricant (iv) is sodium stearyl fumarate (see Example 2).

In a particularly preferred embodiment, API (i) is alogabat; co-processed excipient (ii) is ProSolv SMCC 90; further excipient (iii) is a disintegrant being croscarmellose sodium; and lubricant (iv) is sodium stearyl fumarate (see Example 3).

In a particularly preferred embodiment, API (i) is fenebrutinib; co-processed excipient (ii) is combilac; further excipient (iii) is an acidulant being fumaric acid; and lubricant (iv) is magnesium stearate (see Example 4).

### New Tableting Process

In a further aspect, the present invention provides a mini-batch wise continuous process for manufacturing tablets, comprising the steps of:
(i) feeding an API, a co-processed excipient, and 1-4 further excipient(s) from individual screw feeders each into a mini-batch blender;
(ii) blending the components of step (i) in the mini-batch blender;
(iii) discharging the mini-batch prepared in steps (i) and (ii) into a tablet press;
(iv) compressing the blend from step (iii) into tablet kernels;
(v) repeating steps (i)-(iv) as needed to manufacture the desired batch size; and
(vi) optionally spraying a film coating suspension onto the tablet kernels from step (iv).

In one embodiment, said 1-4 further excipients are selected from fillers, disintegrants, lubricants, and flow agents.

In one embodiment, said fillers are selected from starch, cellulose, sugar alcohols, calcium phosphate and lactose.

In one embodiment, said disintegrants are selected from low substituted hydroxypropyl cellulose, crospovidone, sodium starch glycolate and croscarmellose sodium.

In one embodiment, said acidulant is fumaric acid.

In one embodiment, said lubricants are selected from sodium stearyl fumarate, polyethylene glycol and magnesium stearate.

In one embodiment, said flow agents are selected from colloidal silicon dioxide, polyethylene glycol PEG 6000, fumed silicon dioxide Aerosil® 200, and talc.

In one embodiment, said co-processed excipient is selected from Di-Pac®, Emdex®, Pharmatose®, Sugar Tab®, Pharmaburst 500®, TIMERx®, Ludipress®, Starlac®, Xylitab®, StarCap®, Advantose®, Ludiflash®, Cellactose®, ForMaxx®, Microcelac 100®, Avicel®, ProSolv® SMCC90, Prosolv Easytab®, Combilac®, Startab®, Parteck® ODT, Comprecel SMCC 90, Pharmacel SMCC 90, SANAQ ML 011, and SANAQ SP205.

In a preferred embodiment, said co-processed excipient is selected from Ludipress®, Microcelac®, ProSolv SMCC90®, ProSolv Easytab®, Combilac®, and Startab®.

In a preferred embodiment, said co-processed excipient is selected from Combilac and ProSolv® SMCC90.

In a particularly preferred embodiment, said co-processed excipient is Ludipress®.

In a particularly preferred embodiment, said co-processed excipient is Microcelac®.

In a particularly preferred embodiment, said co-processed excipient is ProSolv SMCC90®.

In a particularly preferred embodiment, said co-processed excipient is ProSolv Easytab®.

In a particularly preferred embodiment, said co-processed excipient is Combilac®.

In a particularly preferred embodiment, said co-processed excipient is Startab®.

In a preferred embodiment, said API is selected from ralmitaront, alogabat, and fenebrutinib.

In a particularly preferred embodiment, said API is ralmitaront.

In a particularly preferred embodiment, said API is alogabat.

In a particularly preferred embodiment, said API is fenebrutinib.

In one embodiment, the rate of the process according to the invention is ≤30 kg, preferably ≤25 kg, more preferably ≤20 kg, more preferably ≤15 kg, most preferably ≤10 kg of tablet kernels per hour.

In one embodiment of the process according to the invention, the mini-batch blender is a high shear blender.

In one embodiment of the process according to the invention, the compressing in step (iv) is direct compressing.

In one aspect, the present invention provides a tablet having a kernel consisting of a blend as described herein above, when obtained from the process according to the invention.

In one aspect, the present invention provides the use of a blend as described herein above in a process according to the invention.

### EXAMPLES

The following examples are provided for illustration of the invention. They should not be considered as limiting the scope of the invention, but merely as being representative thereof.

### Example 1—Continuous Mini-Batch Direct Compression Process

### Equipment

| Process Step | Class | Subclass | Equipment |
|---|---|---|---|
| Feeding | Screw feeders | — | ThreeTec ZD 12 |
| | | | (“small feeders”) and |
| | | | ZD 22 (“large feeder”) |
| Blending | High shear | — | Loedige ploughshare |
| | blender | | mixer 5 L |
| Tablet | Tablet Press | Power Assisted | Korsch XL100 |
| Compression | | | Wipcon or STYL'One |
| | | | Evo |
| Film-Coating | Pan Coating | Perforated | GMPC I |
| | | Coating System | |

### Process

1. Feeding of excipient (i) from the large feeder; and API, excipient (ii), and excipient (iii) from the three small feeders into the mini-batch blender.
2. Blending of the mini-batch in the mini-batch blender.
3. Discharge into the tablet press the mini-batch prepared in steps 1 and 2.
4. Press tablet cores. Perform IPC on the individual tablet weight, hardness, thickness, friability and disintegration time for tablet cores.
5. Repeat steps 1 to 4 as needed to manufacture the desired batch size.
6. Optionally prepare film coating suspension and spray film coat onto tablet cores obtained from step 4. Perform IPC on the average weight, thickness and disintegration time of film-coated tablets.

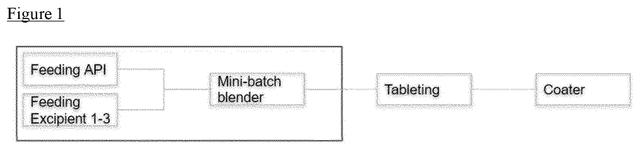

A schematic overview of this process is provided in FIG. 1.

### Example 2—Ralmitaront 150 mg Tablet Formulation

| Component | Function | Amount (mg/tablet) |
|---|---|---|
| Ralmitaront mono | Active | 167.40 mg |
| hydrochloride | Ingredient | (equivalent to 150 mg of |
| | | the free base) |
| ProSolv SMCC90^{a)} | Filler | 287.6 mg |
| Croscarmellose Sodium | Disintegrant | 25.00 mg |
| Sodium Stearyl Fumarate | Lubricant | 20.00 mg |
| Opadry II White | Coating | 20.00 mg |
| Total Tablet Weight | | 520.00 mg |

^{a)}SMCC90 is a commercially available excipient consisting of silicified microcrystalline cellulose.

All excipients used in the formulation are compendial (Ph. Eur. and/or USP/NF) grade.

The tablets may be manufactured according to the continuous process described in Example 1.

### Example 3—Alogabat 20 mg Tablet Formulation

| Component | Function | Amount (mg/tablet) |
|---|---|---|
| Alogabat (dry coated) | Active | 20.00 mg |
| | Ingredient | |
| ProSolv SMCC 90^{a)} | Filler | 97.50 mg |
| Croscarmellose Sodium | Disintegrant | 3.75 mg |
| Sodium Stearyl Fumarate | Lubricant | 3.75 mg |
| Total Tablet Weight | | 125.00 mg |

^{a)}ProSolv SMCC 90 is a commercially available excipient consisting of silicified microcrystalline cellulose.

All excipients used in the formulation are compendial (Ph. Eur. and/or USP/NF) grade.

The tablets may be manufactured according to the continuous process described in Example 1.

### Example 4—Fenebrutinib 200 mg Tablet Formulation I

| Component | Function | Amount (mg/tablet) |
|---|---|---|
| Fenebrutinib | Active | 200.00 mg |
| | Ingredient | |
| Combilac^{a)} | Filler | 376.00 mg |
| Fumaric acid | Acidulant | 200.00 mg |
| Magnesium Stearate | Lubricant | 24.00 mg |
| Total Tablet Weight | | 800.00 mg |

^{a)}Combilac is a commercially available excipient consisting of microcrystalline cellulose, corn starch and lactose monohydrate.

All excipients used in the formulation are compendial (Ph. Eur. and/or USP/NF) grade.

The tablets may be manufactured according to the continuous process described in Example 1.

1. Use of co-processed excipients in continuous manufacturing of solid dosage forms.

2. The use according to claim 1, wherein said solid dosage form is a tablet comprising:
(i) a kernel; and optionally
(ii) a coating.

3. The use according to claim 2, wherein said kernel consists of:
(i) an API;
(ii) 1-4 co-processed excipients; and
(iii) 1-4 further excipients selected from fillers, disintegrants, lubricants, flow agents, and acidulants;
wherein the total number of excipients (ii) and (iii) is ≤5.

4. The use according to claim 3, wherein said kernel consists of:
(i) an API;
(ii) a co-processed excipient;
(iii) a filler, a disintegrant or an acidulant; and
(iv) a lubricant.

5. A blend for continuous direct compression of tablet kernels, consisting of:
(i) an API;
(ii) a co-processed excipient; and
(iii) 1-4 further excipients selected from fillers, disintegrants, lubricants, flow agents, and acidulants.

6. The blend according to claim 5, consisting of:
(i) an API;
(ii) a co-processed excipient;
(iii) a filler, a disintegrant or an acidulant; and
(iv) a lubricant.

7. The use according to claim 3 or 4, or the blend according to claim 5 or 6, wherein said filler is selected from starch, cellulose, sugar alcohols, calcium phosphate and lactose.

8. The use according to any one of claims 3, 4 and 7, or the blend according to any one of claims 5, 6 and 7, wherein said disintegrant is selected from low substituted hydroxypropyl cellulose, crospovidone, sodium starch glycolate and croscarmellose sodium.

9. The use according to any one of claims 3, 4, 7, and 8, or the blend according to any one of claims 5, 6, 7, and 8, wherein said lubricant is selected from sodium stearyl fumarate, polyethylene glycol and magnesium stearate.

10. The use according to any one of claims 3, and 7-9, or the blend according to any one of claims 5 and 7-9, wherein said flow agents are selected from colloidal silicon dioxide, polyethylene glycol PEG 6000, fumed silicon dioxide Aerosil® 200, and talc.

11. The use according to any one of claims 3, 4, and 7-10, or the blend according to any one of claims 5-10, wherein said acidulant is fumaric acid.

12. The use according to any one of claims 1 to 4 and 7 to 11, or the blend according to any one of claims 5 to 11, wherein said co-processed excipient is selected from Di-Pac®, Emdex®, Pharmatose®, Sugar Tab®, Pharmaburst 500®, TIMERx®, Ludipress®, Starlac®, Xylitab®, StarCap®, Advantose®, Ludiflash®, Cellactose®, ForMaxx®, Microcelac 100®, Avicel®, ProSolv® SMCC90, Prosolv Easytab®, Combilac®, Startab®, Parteck® ODT, Comprecel SMCC 90, Pharmacel SMCC 90, SANAQ ML 011, and SANAQ SP205.

13. The use or blend according to claim 12, wherein said co-processed excipient is selected from Ludipress®, Microcelac®, ProSolv SMCC90®, ProSolv Easytab®, Combilac®, and Startab®.

14. The use or blend according to claim 13, wherein said co-processed excipient is selected from Combilac and ProSolv® SMCC90.

15. The use according to any one of claims 3, 4 and 7 to 14, or the blend according to any one of claims 5 to 14, wherein said API is selected from ralmitaront, alogabat, and fenebrutinib.

16. A mini-batch wise continuous process for manufacturing tablets, comprising the steps of:
(i) feeding an API, a co-processed excipient, and 1-4 further excipient(s) from individual screw feeders each into a mini-batch blender;
(ii) blending the components of step (i) in the mini-batch blender;
(iii) discharging the mini-batch prepared in steps (i) and (ii) into a tablet press;
(iv) compressing the blend from step (iii) into tablet kernels;
(v) repeating steps (i)-(iv) as needed to manufacture the desired batch size; and
(vi) optionally spraying a film coating suspension onto the tablet kernels from step (iv).

17. The continuous process according to claim 16, wherein the rate of the process is ≤30 kg, preferably ≤25 kg, more preferably ≤20 kg, more preferably ≤15 kg, most preferably ≤10 kg of tablet kernels per hour.

18. The continuous process according to claim 16 or 17, wherein the mini-batch blender is a high shear blender.

19. The continuous process according to any one of claims 16 to 18, wherein the compressing in step (iv) is direct compressing.

20. A tablet having a kernel consisting of a blend according to any one of claims 5 to 15, when obtained from the process according to any one of claims 16 to 19.

21. Use of a blend according to any one of claims 5 to 14 in a process according to any one of claims 16 to 19.

22. The blend according to any one of claims 5 to 15, having a flowability of >FFc 4-5.

23. The blend according to any one of claims 5 to 15 and 22, having a bulk density of >0.4 g/mL.

24. The blend according to any one of claims 5 to 15, 22 and 23, having a drug load of 1-30% wt/wt, preferably of 2-25% wt/wt, more preferably of 2-20% wt/wt.

25. The invention as described hereinbefore.
